(19) United States
(12) Patent Application Publication
(10) Pub. No.: US 2006/0172408 A1
(43) Pub. Date: Aug. 3, 2006

(54) Device for immobilizing chemical and biochemical species and methods of using same

(76) Inventors: Steven R. Quake, Stanford, CA (US); Emil Kartalov, Pasadena, CA (US)

Correspondence Address: DLA PIPER RUDNICK GRAY CARY US, LLP, 4365 EXECUTIVE DRIVE, SUITE 1100, SAN DIEGO, CA, 92121-2133, US

(21) Appl. No.: 11/002,739

(22) Filed: Dec. 1, 2004

Related U.S. Application Data
(60) Provisional application No. 60/526,162, filed on Dec. 1, 2003.

(51) Int. Cl. C12M 1/34

(52) U.S. Cl. 435/287.2; 435/288.5

(57) ABSTRACT

A substrate is provided that facilitates immobilization of nucleic acid molecules, including DNA molecules and RNA molecules. Also provided is a device that includes the substrate, which, for example, can be a chip. In addition, methods of using the substrate are provided, including, for example, methods of sequencing a DNA molecule anchored to the substrate, and methods for conducting the process of sequencing using such devices.

### CROSS REFERENCE TO RELATED APPLICATIONS

This application claims the benefit of priority under 35 U.S.C. §119(e) of U.S. Ser. No. 60/526,162, filed Dec. 1, 2003, the entire content of which is incorporated herein by reference.

### FEDERAL GOVERNMENT RIGHTS

The invention was made in part with government support under Grant Nos. HG01642 and 5T32-GM07616 awarded by the National Institutes of Health, and DAPRA Grant DAAD19-001-0392. The U.S. Government has certain rights in this invention.

### BACKGROUND OF THE INVENTION

1. Field of the Invention

The present invention relates generally to the field of recombinant DNA technology, and more specifically to devices useful for immobilizing a nucleic acid molecule, for example, a DNA sequencing device to which a DNA sample can be anchored to a substrate and sequencing reactions performed, and to methods for conducting the process of sequencing using such devices.

2. Background Information

A variety of methods have been used to conduct the DNA sequencing. The known methods of DNA sequencing, such as the Sanger method and its subsequent capillary array automation have allowed the sequencing of the consensus human genome. However, this technology has some limitations, especially in terms of cost and read length, which make it difficult to conduct massive comparative genomics studies and aggressive disease-gene discovery.

The limitations of the electrophoretic approach have prompted researchers to work on alternative methods, such as mass spectrometry, base addition with deprotection steps, pyrosequencing, sequencing by hybridization, massively parallel sequencing with stepwise enzymatic cleavage and ligation, polymerase colonies sequencing using nanopores and massively parallel single-molecule sequencing. While some of these methods are promising, none has yet yielded the results that are as good as, or better than, the results provided by the method of electrophoretic separation.

Accordingly, better devices and methods for the DNA sequences are desired. The methods and devices that are needed should be less costly and the sequencing time should be shorter. In addition, it is desirable to have the DNA sequencing devices that are portable and are capable to be integrated with other devices. Unfortunately, better devices and methods having all this advantages have not been described. Thus, need exists to have improved devices for conducting the DNA sequencing.

### SUMMARY OF THE INVENTION

The present invention is based, in part, on the development of a polymer chemistry that allows the formulation of a device useful for immobilizing (anchoring) nucleic acid molecules. The device can be used directly, or can be bound to a substrate, and provides for the specific and tunable derivatization of poly(dimethylsiloxane) (PDMS) for otherwise inhomogeneous arrays. The device bound to a substrate is exemplified by a microfluidic device, which was used to immobilize a DNA molecule for sequencing-by-synthesis.

According to an embodiment of the present invention, a device comprising a multi-layer polymeric structure is provided. The multipolymeric structure includes a graft-copolymer, which includes diacrylated polyglycol, such as diacrylated poly(ethylene glycol), grafted to a second polymer; and a plurality of layers of a polyelectrolyte disposed over the graft copolymer. In one aspect, the device is disposed on (e.g., bonded to) an optically transparent substrate. In another aspect, the device, which is disposed on an optically transparent substrate, comprises a microfluidic device. Such a microfluidic device can include, for example, a first layer that defines a plurality of first channels; a second layer that is bonded to the first layer. In one aspect, the second layer of the microfluidic device further includes a linker, which allows for the immobilization of a target molecule. For example, where the target molecule comprises a biotinylated nucleic acid molecule, the linker can be biotin, which is further contacted with streptavidin, wherein the biotinylated nucleic acid molecule, upon contact with the microfluidic device, is immobilized. In another aspect, the inner surface of each first channel of the microfluidic device is modified to include the multi-layer polymeric structure.

In another embodiment, a microfluidic device for DNA sequencing is provided, the device including a structure disposed on an optically transparent substrate, the structure including a first layer defining one or a plurality of first channels, and a second layer bonded to the first layer, wherein the inner surface of each first channel is modified to include a polymeric layer. In one aspect, a nucleic acid sample is anchored to the polymeric layer. The polymeric layer can be a graft-copolymer comprising diacrylated poly(ethylene glycol) grafted to a second polymer, such as poly(dimethylsiloxane) and can further include a plurality of layers of a polyelectrolyte disposed over the graft copolymer. Further, the microfluidic device can include a linker bound to the polyelectrolyte layer, wherein the linker allows for the immobilization of a target molecule. The linker generally has a specificity for the target molecule such that the target molecule, but not extraneous molecules, are immobilized to the microfluidic device. In one aspect, the linker is specific for a nucleic acid molecule. For example, the linker can be biotin, which is grafted to the microfluidic device surface, and streptavidin, which binds the grafted biotin. Such a linker allows the immobilization of a biotinylated nucleic acid molecule (e.g., biotinylated DNA).

According to another embodiment of the present invention, a method for fabricating microfluidic device is provided. Such a method can be performed by fabricating a first polymeric structure and a second polymeric structure, each structure defining one or a plurality of channels, aligning the first polymeric structure and the second polymeric structure so that the channels in the first polymeric structure are not fluidically connected to the channels in the second polymeric structure, and where the and the channels in the second polymeric structure create a valve action, bonding the first polymeric structure to the second polymeric structure to obtain a fused structure, bonding the fused structure to an optically transparent substrate, and modifying the channel(s) in the first polymeric structure, thereby fabricating the microfluidic device.

In another embodiment, the invention relates to a method of using a device (e.g., a microfluidic device) of the invention for immobilizing a polymer to be sequenced, e.g., a nucleic acid molecule. Such an immobilized nucleic acid molecule conveniently can be examined, for example, by one or more of a sequencing, restriction endonuclease digestion, or hybridization method. The immobilized nucleic acid molecule can be detectably labeled. In one aspect, a plurality of immobilized nucleic acid molecules is provided, thus allowing for high throughput and/or multiplex analysis of the nucleic acid molecules. For example, one or a plurality of immobilized target nucleic acid molecules can be examined using a sequencing-by-synthesis method, wherein the target nucleic acid molecules are contacted with appropriate reagents, including, for example, a polymerase and sequentially with nucleotide triphosphates, wherein each of the nucleotide triphosphates can include a labeled analog (e.g., differentially fluorescently labeled analogs).

In one aspect, the invention provides a method for determining a nucleic acid molecule sequence by performing sequencing-by-synthesis using a device (e.g., a microfluidic device) of the invention. Such a method can be performed, for example, by immobilizing one or more nucleic acid molecule (e.g., 1, 2, 3, 4, 5, etc.), which can be the same or different, to the multi-layer polymeric structure of a device of the invention; contacting the immobilized nucleic acid molecule(s), under conditions suitable for a primer extension reaction, with a polymerase, one or more primers that selectively hybridize(s) to the immobilized nucleic acid molecule(s), thereby obtaining a hybridized primer(s), and at least a first nucleotide triphosphate (NTP) of four nucleotide triphosphates (NTPs), or an analog thereof, for example, a ribonucleotide triphosphate (e.g., ATP, CTP, GTP and UTP), or a deoxyribonucleotide triphosphate (e.g., dATP, dCTP, dGTP and dTTP); and determining whether the hybridized primer is extended by incorporation of the first NTP. Where it is determined that the hybridized primer is extended, the NTP complementary to the nucleotide at the position of the immobilized nucleic acid molecule is identified, thereby determining the nucleic acid molecule sequence. Wherein it is determined that the hybridized primer is not extended, the primer extension reaction is repeated, sequentially, with the second NTP, third NTP, and fourth NTP, as necessary, until the hybridized primer is extended, wherein the extension is indicative of the NTP incorporated and, consequently, the complementary NTP in the “template” immobilized nucleic acid molecule sequence.

Such a method, when performed in a single iteration, allows, for example, the identification of a nucleotide at a position of a single nucleotide polymorphism or of a mutation. Where it is known that the position of the immobilized nucleic acid molecule that is immediately 3′ to the corresponding position of the primer is a polymorphic site (or a mutation site) that contains, for example, dG or dT, a single iteration of the method is sufficient to identify the nucleotide at the position.

In addition, the method can be used in two or more (e.g., 2, 10, 20, 50, 100, 1000, 5000, or more) iterations. Such a method is performed by further contacting the immobilized nucleic acid molecule and the hybridized primer that was extended according to the first iteration as discussed above, with a polymerase, and at least a first NTP of four NTPs, or an analog thereof; and determining whether the hybridized primer is further extended by incorporation of the first NTP, wherein, when the hybridized primer is further extended, an NTP complementary to the nucleotide at the position of the immobilized nucleic acid molecule is identified, thereby determining the nucleic acid sequence, and wherein, when the hybridized primer is not further extended, the further extension reactions are repeated, sequentially, with at least a second NTP, at least a third NTP, and the fourth NTP, until the hybridized primer is further extended.

According to the present methods, the immobilized nucleic acid molecule can be a DNA molecule, in which case the polymerase is a DNA dependent DNA polymerase, or can be an RNA molecule, in which case the polymerase is an RNA dependent DNA polymerase (a reverse transcriptase). The primer extension product generally comprises a DNA molecule, in which case the NTPs or analogs thereof comprise deoxyribonucleotide triphosphates (dNTPs), but also can comprise an RNA molecule, in which case the NTPs or analogs thereof comprise ribonucleotide triphosphates and the polymerase, depending on the immobilized nucleic acid molecule, can be an DNA dependent RNA polymerase or an RNA dependent RNA polymerase.

According to the present methods, the first NTP, second NTP, third NTP, fourth NTP, or a combination thereof can be labeled, for example, with a fluorescence label, radiolabel, luminescent or chemiluminescent label, or paramagnetic moiety, thus facilitating the determination as to whether primer extension has occurred. Further, the inclusion of a label can facilitate automation of the methods such that the methods can be performed with respect to a plurality of nucleic acid molecules, which can be the same (e.g., duplicates, triplicates, etc.) or different (e.g., one or more test nucleic acid molecules and/or one or more controls) or a combination of same and different molecules. As such, the methods can be performed in a high throughput format.

In another aspect, the method for determining a nucleic acid molecule sequence is performed in a multiplex format, wherein one or a plurality of nucleic acid molecules is analyzed in a single reaction, and wherein the multiplex reactions further can be performed in a high throughput format. Such a multiplex method can be performed, for example, by immobilizing a nucleic acid molecule (or each of a plurality of nucleic acid molecules, independently) to each of five positions on the multi-layer polymeric structure of the device of the invention; and contacting each position, under conditions suitable for a primer extension reaction, the immobilized nucleic acid molecule with a polymerase, a primer that selectively hybridizes to the immobilized nucleic acid molecule, thereby obtaining a hybridized primer, and one of four NTPs, or an analog thereof, wherein each of the five positions is contacted with one of the NTPs; and determining at which of the five positions the hybridized primer is extended by incorporation of an NTP, wherein the position is indicative of the NTP incorporated into the hybridized primer, which is complementary to the nucleotide at the position of the immobilized nucleic acid molecule is identified, thereby determining the nucleic acid sequence. Such a method can further include contacting the positions at which the hybridized primer was not extended (i.e., the other four positions) with an NTP corresponding to the NTP incorporated into the hybridized primer, wherein the NTP is incorporated into the hybridized primer, thus extending the hybridized primers in each of the five positions to the same extent; and sequentially repeating the multiplex reaction to determine the next position in the immobilized nucleic acid molecule. By combining the devices of the invention and performing the reactions as indicated, sequences up to several thousand (e.g., 2000, 3000, 4000, 5000, 6000, 7000, 8000, or more) nucleotides in length can be determined.

### BRIEF DESCRIPTION OF THE DRAWINGS

FIGS. 1A, 1B and 1C show schematically a device for DNA sequencing according to an embodiment of the present invention.

FIGS. 2A-2E illustrates schematically a process for fabricating a device according to an embodiment of the present invention.

FIGS. 3A and 3B also illustrate schematically a process for fabricating a device according to an embodiment of the present invention.

FIG. 4 also illustrates schematically a process for fabricating a device according to an embodiment of the present invention.

FIG. 5 shows schematically an assembly including a plurality of DNA sequencing devices according to an embodiment of the present invention.

FIG. 6 shows schematically results of DNA sequencing using a device and a method according to embodiments of the present invention.

### DETAILED DESCRIPTION OF THE INVENTION

U.S. patent application Ser. No. ______, Quake et al., filed Dec. 1, 2004 as attorney docket HEL-918, is herein incorporated by reference in its entirety.

The following terminology, definitions, and abbreviations apply:

The term “microfluidic” refers to substrate having a fluid passage with at least one internal cross-sectional dimension that is less than 500 micrometers and typically between about 0.1 micrometers and about 500 micrometers. Additionally, “microfluidics” refers to, including without limitation (a) microfluidics technology that is, has or uses substrates (e.g., chips) having at least one well, via or channel with a feature size of 500 microns or less for transporting fluids. One way of fabricating microfluidic devices includes multi-layer soft lithography, such as described in U.S. Pat. No. 6,793,753, herein incorporated by reference in its entirety.

The term “substrate” refers to a planar base layer of a dielectric material. The substrate can be homogenous, and can include one or a plurality (e.g., 2, 3, 4, 5, 6, or more) of channels.

The term “channel” refers to a groove in a substrate that allows the contained passage of a fluid. Generally, the channel is a microfluidic channel, wherein fluid elements are dimensioned such that flow therein is substantially laminar.

The term “photoresist” refers to a radiation-sensitive material. The photoresist can be any radiation-sensitive material, including, for example, a material sensitive to ultraviolet (UV) radiation.

The term “photomask” or “mask” refers to a photolithographic device used to block the exposure of photoresist to UV radiation in selected areas.

The term “poly(dimethylsiloxane)” or “PDMS” is used herein to include both oligomers and polymers derived from monomeric dimethylsiloxane, the polymers and oligomers having the general formula: 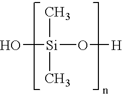
For PDMS oligomers, n in the above formula can be between 2 and 20; for PDMS polymers, x can be more than 20.

The term “monomeric dimethylsiloxane” refers to a compound having the formula:

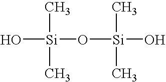

The term “diacrylated poly(ethylene glycol)” or “DAPEG” refers to an oligomer or polymer having the general formula: 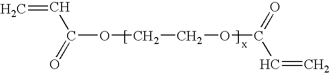
For DAPEG oligomers, x in the above formula can be between 2 and 20; for DAPEG polymers, x can be more than 20.

“Biotin” is the compound having the formula:

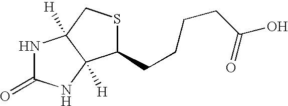

“Streptavidin” is a protein that is produced by Streptomyces avidinii and capable of binding biotin. Streptavidin is secreted into the culture broth in which the bacterium is grown.

The term “biotinylated nucleic acid molecule” refers to a conjugate of biotin and a nucleic acid molecule. The nucleic acid molecule can be DNA, RNA, a DNA/RNA hybrid, or analogs thereof. The biotin moiety can be conjugated to the nucleic acid using chemical methods, or can incorporated into the nucleic acid molecule enzymatically using, for example, a polymerase and a nucleotide analog comprising the biotin moiety.

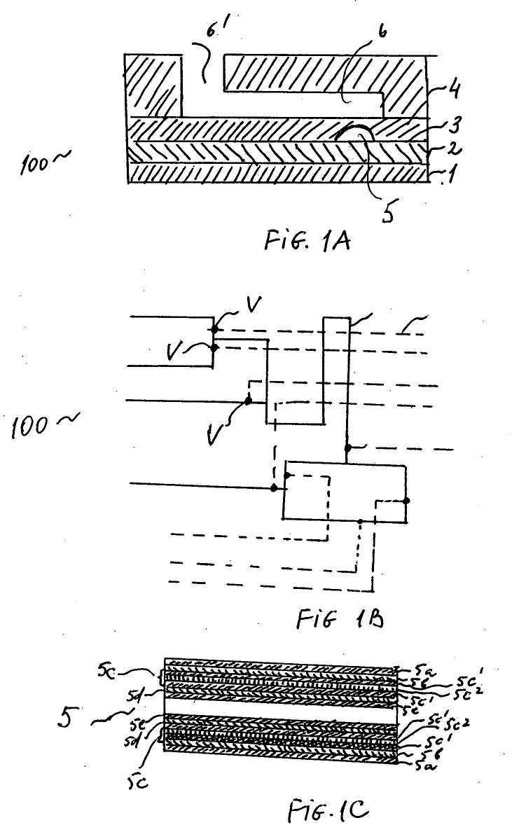

According to one embodiment of the invention, a method for fabricating a microfluidic device for DNA sequencing is provided. The device can be described with the reference to FIGS. 1A-1C.

FIG. 1A is a schematic illustration showing a cross-section of a device of the invention 100 according to an embodiment of the present invention. FIG. 1B depicts schematically the same device shown as top view. The device 100 includes a substrate 1, which can be made of an optically transparent material such as glass. On top of the substrate 1 there is disposed an adhesive polymer layer 2, bonded to the substrate 1. The adhesive polymer layer 2 can be made of poly(dimethylsiloxane) (PDMS). Those having ordinary skill in the art can select another polymer to make the polymer layer 2. Examples of alternative polymers suitable for making the adhesive polymer layer 2 include poly(methylmethacrylate) or poly(urethane). The adhesive polymer layer 2 can have thickness between about 10 micrometers and 20 micrometers.

Over the polymer adhesive layer 2, there are disposed the flow layer 3 and the control layer 4. As can be seen from FIG. 1A, the flow layer 3 can be bonded to the substrate 1 via the polymer adhesive layer 2. The flow layer 3 can have thickness between about 30 micrometers and 40 micrometers, and the control layer 4 can have thickness between about 4 millimeters and 6 millimeters, for example, about 5 millimeters. The flow layer 3 and the control layer 4 are bonded to each other. The procedure that can be used to accomplish such bonding is described subsequently in the present application. Each of the flow layer 3 and the control layer 4 can be made of a polymer, such as PDMS. Alternatively, those having ordinary skill in the art can select another polymer to make the flow layer 3 and the control layer 4; for example, poly(methylmethacrylate) or poly(urethane) can be used, if desired.

Each of the flow layer 3 and the control layer 4 can define a plurality of channels. For simplicity and for illustrative purposes only, FIG. 1A depicts only one channel in the flow layer 3 (the flow channel 5) and only one channel in the control layer 4 (the control channel 6); however, it should be understood that each of the flow layer 3 and the control layer 4 can include many channels, for example, between 2 and 50. The dimensions and shapes of each flow channel 5 and each control channel 6 can vary depending on manufacturing conditions and design. In one embodiment, the channels can have the height between about 8 micrometers and 12 micrometers, for example, about 10 micrometers, and the width along the longest linear dimension (e.g., in case of cylindrical channels, along the diameter), between about 60 micrometers and 150 micrometers, for example, about 100 micrometers. The shape of the flow channels 5 and the control channels 6 can vary; for example, flow channels 5 can be semi-circular in cross-section. As can be seen from the top view (FIG. 1B), flow channels 5 partially overlap control channels 6 creating valve action at points V.

The inner surface of each flow channel 5 can be modified to form a multilayer structure, a cross section of which is shown by FIG. 1C. In an exemplary embodiment shown by FIG. 1C, the inner surface of a flow channel 5, which is substantially cylindrical, is made of PDMS (shown as layer 5a), includes a layer 5b of diacrylated polyglycol, such as poly(ethylene glycol) (DAPEG) that is grafted to the PDMS surface. The procedure that can be used to graft DAPEG to PDMS is described subsequently in the present application. Examples of other diacrylated polyglycols that can be used include straight-chained or branched polyglycols, e.g., poly(propylene glycol), poly(butylene glycol), and the like, so long as the viscosity and other physical properties of the diacrylated polyglycol allows its penetration into flow channel 5.

A polyelectrolyte structure 5c can be formed over the DAPEG layer 5b. The polyelectrolyte structure 5c can include a plurality of negative sub-layers 5c¹ and positive sub-layers 5c² alternating as shown by FIG. 1C, where the outermost sub-layer of the polyelectrolyte structure 5c is the negative sub-layer 5c¹. A variety of materials can be used to form the negative and positive sub-layers. For example, poly(acrylic acid) can be used to form the negative sub-layers 5c¹. Poly(ethyleneimine) or poly(allylamine) can be used to form the positive sub-layers 5c². Those having ordinary skill in the art can determine the total number of sub-layers in the polyelectrolyte layer 5c. In one embodiment, the polyelectrolyte structure can include a total between 2 and 100 sub-layers, for example, between 10 and 50, such as between 10 and 16 sub-layers. In one embodiment, each sub-layer can have a thickness between about 4 and 6 nanometers, for example, about 5 nanometers. A thin layer of biotin 5c can be deposited over the polyelectrolyte structure 5c, followed by a thin layer of streptavidin 5e, deposited over the biotin layer 5d. The procedures that can be used to form the biotin layer 5d and the streptavidin layer 5e are described below.

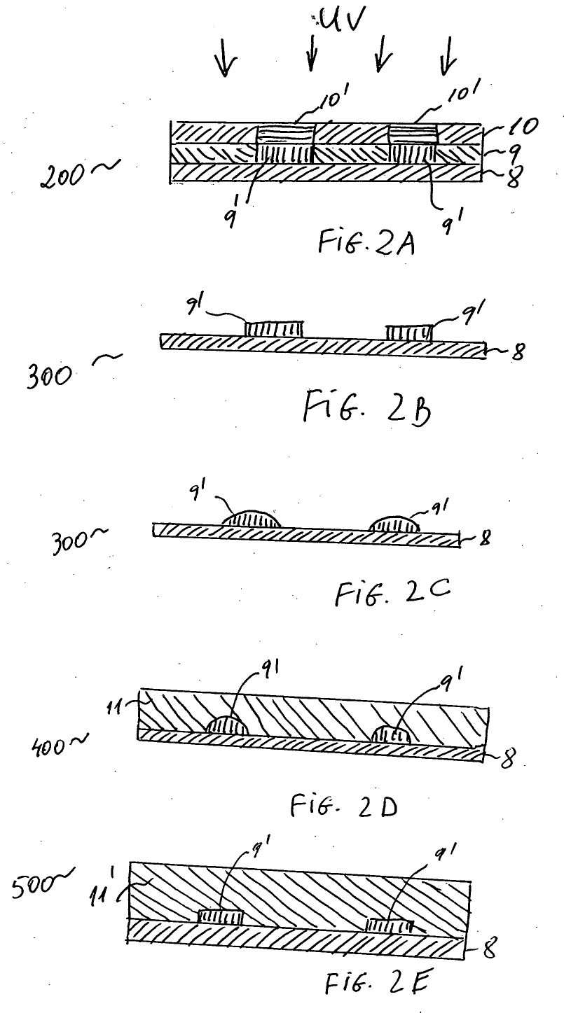

According to one embodiment of the invention, a method for fabricating a microfluidic device for DNA sequencing shown by FIGS. 1A-1C is provided. The method includes a plurality of steps that can be described with the reference to FIGS. 2A-2E, FIG. 3A, 3B and FIG. 4.

To make a device for DNA sequencing shown by FIGS. 1A-1C, two molds can be fabricated first. With reference to FIG. 2A, the initial material for fabrication can be a silicon wafer 8. The silicon wafer 8 can be made of either crystalline or amorphous silicon. The wafer 8 can have the thickness between about 100 micrometers and 1 millimeter. The silicon wafer 8 can be thoroughly cleaned in preparation for further processing. Any suitable method of cleaning used in the semiconductor fabrication technologies can be employed, such as multiple washing in de-ionized water or in a solvent, e.g., ethanol or acetone, followed by drying.

A layer of photoresist 9 can be then deposited over one side of the wafer 8. Any photoresist known in the art of semiconductor fabrication can be used for forming the layer 9. Either positive or negative photoresist can be used. Areas 9′ on the photoresist layer 9 indicated where there ridges in the mold will be, as discussed below.

The photoresist layer 9 can be prepared by dissolving a polymer, a photosensitizer, and a catalyst in a solvent to make a photoresist solution, followed by depositing the photoresist solution over the silicon wafer 8, and baking, to make the layer 9. Any method known in the art of semiconductor fabrication can be used for depositing the photoresist solution. For example, the spin coating method can be used, typically involving spinning speeds of between about 1,000 and about 5,000 revolutions per minute, for about 30 to 60 seconds, resulting in the thickness of wet photoresist layer 9 ranging between about 1 μm and 10 to 50 μm, depending on the particular photoresist that is selected.

In the photoresist solution described above, the mass concentration of the polymer can be between about 40% and about 50%, the mass concentration of the photosensitizer can be between about 1 % and about 5%, the mass concentration of the catalyst can be between about 5% and about 10%, the balance comprising a suitable solvent. Any polymer, for example, poly(methyl methacrylate) (PMMA) can be used for making the photoresist solution described above. Those having ordinary skill in the art can selected another polymer, if desired. Representative, non-limiting examples of photosensitizers that can be used include benzophenone or xanthine. Representative, non-limiting examples of catalysts that can be incorporated into the photoresist layer 9 include salts of sulfonium or salts of iodonium. For example, when the polymer used in the photoresist layer 9 is PMMA, the photosensitizer can be benzophenone and the catalyst can be diphenyliodonium chloride. The solvent to be used in fabricating the photoresist layer 9 can be selected by those having ordinary skill in the art depending on the particular polymer, photosensitizer, and catalyst that are used in the photoresist layer 9.

Following the formation of the photoresist layer 9, a photomask 10 can be applied over the photoresist layer 9 in such as way as to cover a portion of the photoresist layer 9, while leaving another portion of the photoresist layer uncovered, to form the structure 200 shown on FIG. 2A. The mask 10 can be applied using standard techniques and materials used in semiconductor fabrication industry and known to those having ordinary skill in the art. For example, the mask can be a glass plane having patterned emulsion or metal film on one side. Areas 10′ of the photomask layer 10 overlap the photoresist areas 9′.

Ultra violet (UV) radiation can then be directed at the photoresist layer 9 as shown by FIG. 2A. Wavelength of the UV radiation can be about 365 nm, and the duration of the UV exposure can be between about 1 minute and about 5 minutes, for example, about 3 minutes. The UV radiation can be generated by any standard commercially available source, to be selected by those having ordinary skill in the art.

Following the exposure to the UV radiation, the entire photomask layer 10 and portions of the negative photoresist layer 9 are destroyed and removed, as known to those having ordinary skill in the art, leaving the structure 300, including the wafer 8 and the ridges 9′, which are the remainder of the photoresist layer, as shown by FIG. 2B. The structure 300 is then baked, for example, at about 100° C. for about 30 minutes, to shape ridges 9′ to have the semi-circular form shown by FIG. 2C.

The process of fabrication of the second mold is similar to the process of fabrication of the first mold described above and shown schematically by FIGS. 2A-2C, except no annealing is performed in the process of fabrication of the second mold. The same wafer, photoresist and photomask materials can be used as those used for fabrication of the first mold. As a result, the second mold having the structure like that shown by FIG. 2B can be obtained.

The first and the second molds made as described above can then be exposed to the environment comprising trimethylchlorosilane, for example, by being placed into a chamber containing saturated vapor of trimethylchlorosilane for between about 2 and 3 minutes at room temperature, resulting in deposition of a thin layer of trimethylchlorosilane on both molds. The trimethylchlorosilane is deposited to ensure that PDMS can be smoothly peeled off, as described later.

PDMS can be then deposited over the thin layer of trimethylchlorosilane. To deposit a layer of PDMS, a composition comprising a blend of monomeric and oligomeric dimethylsiloxane (“a siloxane system”) and a catalyst can be applied onto the mold. The catalyst can be platinum-based and can include a cross-linking agent such as a vinyl compound. The mixtures having different siloxane-to-catalyst ratios can be used for the first and the second mold.

To form the PDMS layer on the first mold, a mixture containing about 1 mass part of the blend of the catalyst and crosslinker per 20 mass parts of the siloxane system can be used. To form the PDMS layer on the second mold, a mixture containing about 1 mass part of the blend of the catalyst and crosslinker per 5 mass parts of the siloxane system can be used.

One way to prepare a siloxane/catalyst mixture can be by using a two-package product, where the first package contains a siloxane system, and the second package contains an appropriate catalyst and cross-linker. After the two packages have been mixed, the catalyst causes rapid polymerization of the siloxanes in the system, leading to formation of PDMS. Those having ordinary skill in the art can select a suitable system and mix the siloxane system and the catalyst in desired ratio prior to use. There exist many commercially available 2-package products that can be used. Specific examples of 2-package siloxane/catalyst products that can be utilized include Sylgard 184 or General Electric's RTV product. After the siloxane system/catalyst mixture having the desired siloxane-to-catalyst ratio has been prepared, the mixture can be applied over the first mold and the second mold, to form a PDMS layer over the mold.

For the first mold, the siloxane system/catalyst mixture can be applied over the mold using the spin coating method. The first mold can be placed on a spinner, silicon side down, to apply the siloxane mixture only to the side having the ridges 9′. The spinning speed can be between about 1,000 and about 5,000 revolutions per minute, for example, about 2,500 revolutions per minute, and the duration of spinning can be about 1 minute resulting in the formation of the PDMS layer 11 having thickness between about 30 micrometers and 40 micrometers, as shown by FIG. 2D. As can be seen from FIG. 2D, the structure 400 includes the PDMS layer 11 covering the rounded ridges 9′.

For the second mold, the siloxane system/catalyst mixture can be applied over the mold by pouring between about 40 and 50 grams of the system over the second mold, for example, in a Petri dish, resulting in the formation of the PDMS layer 11′ having thickness of about 5 millimeters. As can be seen from FIG. 2E, the structure 500 includes the PDMS layer 11′ covering the ridges 9′.

The first and the second mold can be baked at about 80° C. for about 30 minutes, followed by cooling at room temperature for about 5 minutes, resulting in solidifying both PDMS layers 11 and 11′, followed by further processing. The PDMS layer 11′ can be peeled off from the second mold, to obtain the structure 600 (FIG. 3A). The structure 600 is entirely made of PDMS layer 11′ defining the plurality of channels 6. Using a vertical press and a 20 gauge needle, orifices having the diameter of about 625 micrometers (not shown) can then be formed by puncturing the structure 600 substantially through the middle of channels 6. In the emerging device, these orifices can become ports which can serve to supply pressure to the channels 6 from the outside world to produce valve action at the wider overlapping regions of flow channels 5 and control channels 6. The punctured structure 600 can then be washed in ethanol and dried to remove the debris.

The structures shown by FIGS. 2D and 3A can then be assembled as shown by FIG. 3B to form the structure 700. To assemble the structure 700, the structure 600 having the orifices punctured in it, can than be placed over the PDMS layer 11 of the first mold, and the ridges 9′ can be aligned with the channels 6 in the structure 700 so that selected regions overlap to form future valves. A stereoscope can be used for aligning. Following the alignment, the entire structure 700 can be baked at about 80 oC for about 60 minutes, resulting in complete fusion of the PDMS layers 11 and 11′, followed by cooling at room temperature for about 5 minutes.

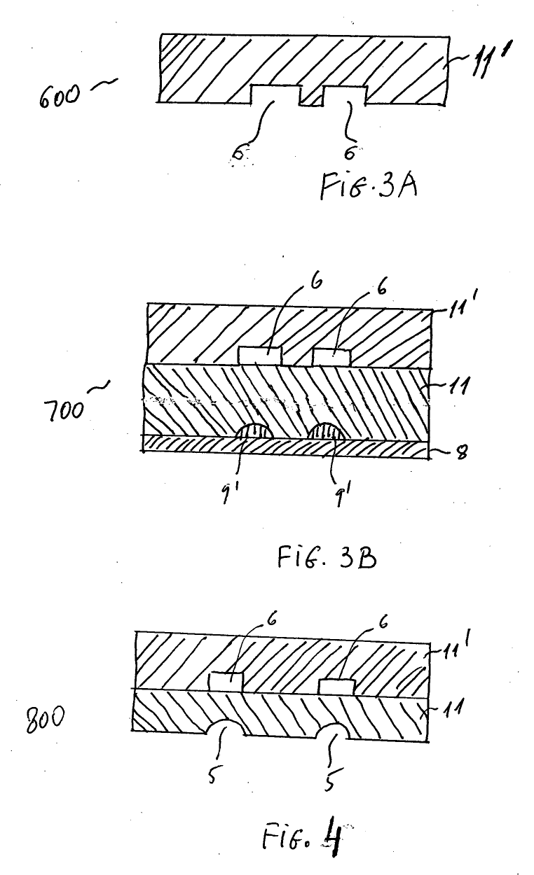

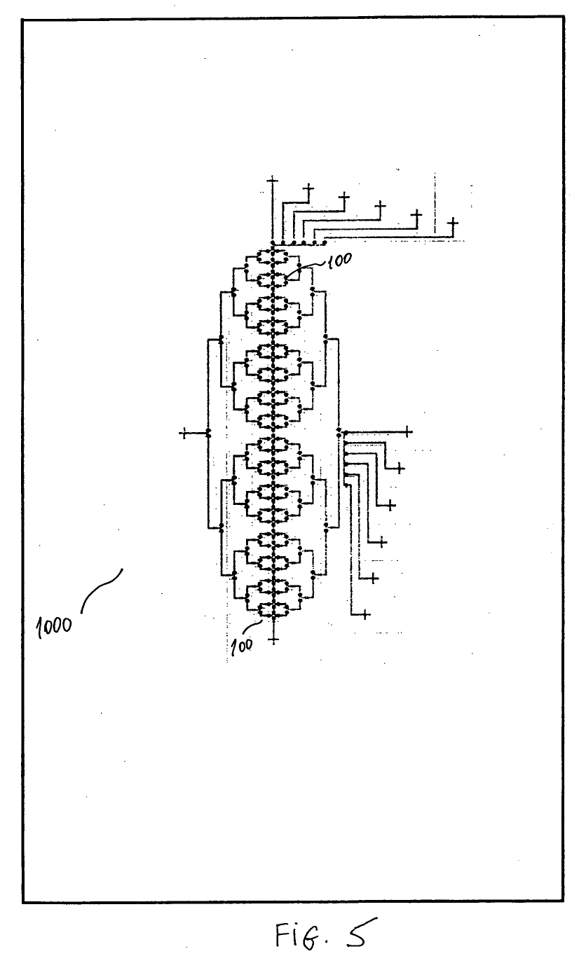

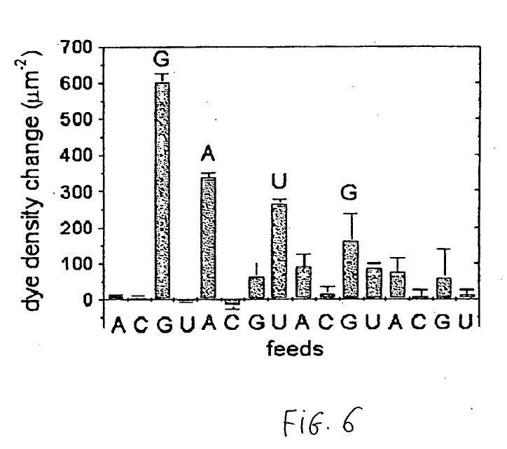

Using the tweezers, the fused PDMS layer comprising PDMS layers 11 and 11′ can be detached from the wafer 8, to form the structure 800 the side view of which is shown by FIG. 4. Orifices can be then punctured at the back side of the layer 11 and completely through it (not shown) and through the flow channels 5, using the 20 gauge needle and press described above. These orifices connect the flow channels 5 to the outside world and become ports, through which reagents can be supplied into the chip. After the orifices have been made, the flow layer can then be washed in ethanol and dried to remove the debris.

Cover slips can be now prepared. Cover slips can include optically transparent slides, e.g., glass slides, and an adhesive layer applied on one side of the slides. The thickness of the glass slides can be about 125 micrometers, and the thickness of the adhesive layer can be between about 10 and 20 micrometers. The adhesive layer can comprise PDMS and can be made by applying the above described siloxane system/catalyst mixture having the siloxane-to-[catalyst+cross-linker] ratio of about 5:1. The adhesive layer can be applied over the glass slides by spin coating at about 5,000 revolutions per minute for about 1 minute, followed by baking at about 80° C. for about 30 minutes.

The structure 800 having flow channels 5 and the control channels 6, can then be placed over the adhesive layer of the cover slip, the layer 11′ facing up. The entire structure can then be baked at about 80° C. for about 120 minutes, resulting in the final microfluidic device 100 shown by FIG. 1A. It is noteworthy, that in the final product the control channels 6 only partially overlap the flow channels 5 and control channels 6 and flow channels 5 are not fluidically connected, yet the control channels 6 and the flow channels 5 can create a valve action where they overlap at points V (FIG. 1B).

Following the process of fabrication of the microfluidic device, the inside surface of flow channels can be treated and modified, to obtain a multi-layer structure shown by FIG. 1C. Layers 5a, 5b, 5c, 5d, and 5e can be successively built, and the process can be controlled by applying pressure via the pressure port 6′ (FIG. 1A). If desired, only a portion of the inside surface of flow channels can be similarly treated.

To conduct the surface modification, a DAPEG solution can be prepared first. The solution can contain DAPEG and platinum-based catalyst, e.g., dihydrohexachloroplatinate, in a volumetric ratio of about 200:1. The solution can be introduced inside the flow channel using a syringe. About 10 nanoliters of the solution can be placed inside the flow channel. The device 100 can then be baked at about 80° C. for about 120 minutes, resulting in the formation of DAPEG layer 5b grafted to the PDMS inner surface of the flow channel. After cooling down to room temperature, the excess DAPEG mixture is washed out of the flow channels, for example using deionized water.

After the process of grafting is complete, a polyelectrolyte multi-layer 5c (FIG. 1C) can be built. As mentioned above, the negative sub-layer 5c¹ is the outermost sub-layer of the polyelectrolyte multi-layer 5c. Poly(acrylic acid) can be used for making the negative sub-layers, and poly(ethyleneimine) or poly(allylamine) for the positive sub-layers, as discussed above. The duration of application of each sub-layer can be about 2 minutes, at a pressure of about 5 psi. The sub-layer then self-assemble electrostatically, at room temperature, so long as the solutions are at pH around 7, since the pKa of the carboxyl group is about 4 and of the amino group is about 10. Each of the sub-layers 5c¹ and 5c² can be applied in an alternating manner, with flushing with de-ionized water for about 2 minutes after each sub-layer has been applied.

After the polyelectrolyte layer 5c has been deposited, biotin linker layer 5d can be applied over the polyelectrolyte layer 5c. The linker has an aminogroup at the one end and biotin at the other. When activated, the amino group reacts with the carboxyl group on the outermost negative layer to form a peptide bond and thus graft the biotin to the surface. Commercially available biotin linker (e.g. Biotin-EZ-Link kit from Pierce) can be fed into the flow channel 5 for about 2 minutes, followed by the incubation period of about 10 minutes. The cycle of feeding biotin linker and incubation can be repeated at least twice. Following the application of biotin linker, streptavidin layer 5e can be formed. Streptavidin can be applied in a buffer solution, under the same conditions as used for applying biotin. Those having ordinary skill in the art can determine the amount of biotin and streptavidin that need to be deposited to form the layers 5d and 5e, respectively.

If desired, an assembly 1000 including a plurality of devices 100 can be made, as shown by FIG. 5. A 32-chamber device is illustrated by FIG. 5, which shows only the flow channels and the valves. The assembly would allow conducting the sequencing of many DNA samples simultaneously or consecutively, as needed, thus making the process of sequencing more efficient and flexible.

Following the process of modification the flow channels 5, as described above, the microfluidic device 100 is ready for polymer sequencing, e.g., DNA sequencing, protein sequencing, sugar sequencing, and the like. Briefly, the process of sequencing can be characterized as sequencing by synthesis, as this term is understood by those having ordinary skill in the art. By way of illustration, with regard to DNA sequencing, the process includes exposing a primed DNA template to a mixture of a known type of standard nucleotide, its fluorescently tagged analog, and DNA polymerase. If the tagged nucleotide is complementary to the template base next to the primer's end, the polymerase can extend the primer with it and fluorescence signal can be detected after a washing step. Iteration with each type of nucleotide can reveals the DNA sequence. The average read length is currently 3 base pairs (bp) in these microfluidic devices.

To conduct the process of sequencing, a sample of DNA to be sequenced can be first modified by biotinylation (i.e., binding biotin to the sample), to obtain a biotinylated sample of a DNA thereby. The process of biotinylation can be conducted using conventional techniques known to those having ordinary skill in the art. A biotinylated sample of a DNA can then be introduced into the flow channel 5 where it can bind to streptavidin. Nucleotides, their fluorescent analogs and polymerase can be then fed one type of nucleotide at a time into the flow channel 5.

A microfluidic device of the invention provides a means to immobilize one or a plurality of nucleic acid molecules to the multi-layer polymeric structure surface of the device such that the nucleic acid molecule(s) conveniently can be analyzed. As disclosed herein, immobilization of a nucleic acid molecule is stable, for example, to contact with reagents, including to sequential passage of solutions over the nucleic acid molecule (see, also, Kartalov and Quake, Nucl. Acids Res. 32:2873, 2004, which is incorporated herein by reference). In various aspects, the device can contain sites for substantially irreversible binding of nucleic acid molecules, or sites for reversible binding of the nucleic acid molecules. For example, the device can contain linker molecules such as biotin as a component of the multi-layer polymeric structure surface of the device. Such a device can be contacted with avidin or streptavidin, then with a nucleic acid molecule of interest that is biotinylated at a terminus of interest (e.g., at the 3′ terminus or the 5′ terminus). Upon contact of the biotinylated nucleic acid molecule with the multi-layer polymeric structure surface, the biotin moiety binds streptavidin, thereby essentially irreversibly binding (Kd approx. 10⁻¹⁵ M) the nucleic acid molecule to the device.

A microfluidic device also can provide a means for irreversibly immobilizing a target nucleic acid molecule. For example, the device can contain an irreversibly bound oligonucleotide linker that can be used to immobilize a target nucleic acid molecule via hybridization. Preferably, the linker oligonucleotide does not contain a sequence of interest in the target molecule or a sequence that is complementary to the nucleotide sequence of interest of a target molecule. By way of example, the linker can be an oligodeoxadenosine (oligo-dA) molecule, which can hybridize to an oligodeoxythymidine (oligo-dT) sequence of a nucleic acid molecule of interest. Such an oligo-dT sequence of a nucleic acid molecule of interest can be, for example, an oligo-dT sequence that is added to a first strand DNA synthesized by reverse transcription of an mRNA molecule using oligo-dT as a primer, or can be an oligo-dT sequence that is engineered to a terminus of a nucleic acid molecule of interest.

A target nucleic acid molecule immobilized to a microfluidic device of the invention can be examined in any of various ways, as desired. For example, the target molecule can be contacted with a primer, nucleotides and/or nucleotide analogs, and a polymerase, whereby a primer extension reaction can proceed. In one aspect, such a method can be used to perform sequencing-by-synthesis of all or a portion of the target nucleic acid molecule (e.g., a sequence containing or suspected of containing a mutation, a single nucleotide polymorphism, or the like). An example of sequencing-by-synthesis on a microfluidic device is provided in Example 2, wherein a primer, a nucleotide and its corresponding fluorescently labeled analog, and polymerase were contacted with the target nucleic acid molecule under conditions such that the polymerase can extend the primer if the nucleotide (and analog) is complementary to the nucleotide in the template target molecule. If the nucleotide is complementary, a portion of the extension product will incorporate the nucleotide analog, which can be detected using a fluorescence detector. If no fluorescence is detected, the reagents are washed out from the position of the target molecule, and a second reaction mixture containing a different nucleotide (and fluorescently labeled analog) are contacted with the target. If no fluorescence is detected, the reagents are washed from the target, and a third reaction mixture containing a different nucleotide (and analog) are contacted with the target, these steps being repeated with each of the four nucleotides (adenosine, cytidine, guanidine, and thymidine) and corresponding fluorescent analog until fluorescence is detected, wherein the fluorescence provides an indication of the nucleotide at the particular position. Upon identifying a first nucleotide, the steps are repeated until the second, third, etc, (as appropriate) are identified. For convenience, the nucleotide analogs can contain different fluorescent labels, which preferably have non-overlapping excitation and/or emission spectra, thus facilitating identification of a particular incorporated nucleotide and further allowing for the sequencing to be performed in a multiplex and/or high throughput format.

An immobilized target molecule also can be analyzed by contacting the target molecule with a restriction endonuclease (e.g., a restriction endonuclease that selectively cleaves a methylated (or unmethylated) recognition site), whereby detection of cleavage (or lack of cleavage) of the target molecule provides information about the target nucleic acid molecules (e.g., that a CpG island is methylated or is unmethylated). According to this method, the immobilized target molecule can be labeled, for example, at the terminus distal from that bound to the device, wherein, upon cleavage by the restriction endonuclease, a sequence comprising the label moiety is released from the device and can be removed from the sample. Upon contact with the endonuclease, the reaction solution can be removed from the position of the target molecule, wherein detection of the cleavage event can be monitored by detecting the loss of label from the position of the immobilized target nucleic acid molecule, or by detecting the presence of the label in removed reaction solution.

A target molecule can be labeled with any moiety that conveniently can be detected. Labels for nucleic acid molecules are well known and include, for example, radionucleotides, fluorescent molecules, paramagnetic molecules, luminescent or chemiluminescent molecules, and tags such as biotin. A target nucleic acid molecule also can be labeled with a fluorescence resonance energy transfer (FRET) pair, wherein a change in fluorescence occurs upon cleavage of the target molecule due to a change in proximity of the FRET pair. The FRET pair can be incorporated into the target molecule in appropriate proximity to each other, or can be provided as a hybridizing oligonucleotide that can selectively bind to the target molecule. The FRET pair can be a first fluorescent molecule with an emission energy that overlaps the excitation energy of a second fluorescent molecule, wherein, when the molecules are in proximity, the second fluorescent molecule fluoresces, and wherein the fluorescence is lost upon separation of the first and second fluorescent molecules. The FRET pair also can be a fluorescent molecule and a quencher that quenches the fluorescent energy of the fluorescent molecule, wherein, when the molecules are in proximity, fluorescence is quenched, and wherein fluorescence can be detected when the quencher is separated from the fluorescent molecule.

The following examples are intended to illustrate but not limit the invention.

### EXAMPLE 1

### Preparation of a Microfluidic Device

This example illustrates the process of fabricating a microfluidic device having a surface suitable for immobilization of a nucleic acid molecule.

As disclosed herein, the microfluidic device is useful in the construction of a nucleic acid sequencing device. Such a device is exemplified by the DNA-sequencing device shown by FIGS. 1A-1C.

The following reagents were used for fabrication. Hexamethyldisilazane (HMDS) from ShinEtsuMicroSi, Phoenix, Ariz. was used. Photoresist 5740 from MicroChem Corp., Newton, Mass. was used. Tetramethylchlorosilane from Aldrich was used. Poly(dimethylsiloxane) (PDMS) Sylgard 184 from Dow Corning, K. R. Anderson, Santa Clara, Calif. was used. Diacrylated poly(ethylene glycol)(DAPEG) SR610 from Sartomer, Exton, Pa. was used. The Pt catalyst was hydrogen hexachloroplatinate from Aldrich. The polyelectrolytes that were used were polyethyleneimine (PEI) from Sigma and polyacrylic acid from Aldrich. Biotin from a kit from Pierce was used. Streptavidin that was used was Streptavidin Plus from Prozyme, San Leandro, Calif. The buffer was Trisb that is Tris 10 mM (NaCl 10 mM), pH 8.

The procedure that was used for fabrication of microfluidic device generally corresponded to the above-described process, with the following modifications. PDMS microfluidic devices with integrated micromechanical valves were built using soft lithography. Silicon wafers were exposed to HMDS vapors for about 3 minutes. Photoresist 5740 was spun at about 2,500 rpm for about 60 seconds on a Model WS-400A-6NPP/LITE spinner from Laurel Technologies Corp. The wafers were baked at about 105° C. for about 90 seconds on a hotplate. UV exposure through black-and-white transparency masks was done at about 180 mW/cm² for about 25 seconds on a mask aligner (Karl Suss America Inc., Waterbury, Vt.).

The molds were then developed for about 3 minutes using a 2401 MicroChem developer. The flow layer molds were baked at about 100° C. for about 30 minutes on a hotplate to melt the photoresist and round the flow channels. The molds were characterized on Alpha-Step 500 apparatus from KLA-Tencor, Mountain View, Calif. The channel height was between about 9 micrometers and 11 micrometers, while the main flow channel width was between about 95 micrometers and 105 micrometers. The profile of the control channel was rectangular, while that of the flow channel was approximately parabolic. Except for the height measurements and the flow channel rounding, the mold fabrication was conducted in a class 10,000 clean room.

Molds were exposed to the TMCS vapors for about 3 minutes. PDMS was mixed with the [catalyst+crosslinker] at about 5:1 and about 20:1 ratios. These two samples were degassed using HM-501 hybrid mixer and cups from Keyence Corp., Long Beach, Calif. Then, about 35 grams of the 5:1 mixture was poured onto the control mold in a plastic Petri dish wrapped with aluminum foil. About 5 grams of the 20:1 mixture was spun over the flow mold at about 2,500 rpm for about 60 seconds on Spincoater P6700 from Specialty Coating Systems, Indianapolis, Ind. Both molds were baked in an oven at about 80° C. for about 30 minutes. The control layer was then taken off its mold and cut into chip pieces. Control line ports were punched using a 20-gauge Luer-stub adapter from Beckton-Dickinson, Franklin Lakes, N.J. Control layer pieces were washed with ethanol, blown dry, and aligned on top of the flow layer under a stereoscope, followed by baking in an oven at about 80° C. for about 1 hour.

Chip pieces were then cut out and peeled off the flow layer mold. Flow line ports were punched with the same 20-gauge Luer-stub adapter. Meanwhile, 5:1 PDMS mixture was spun at about 5,000 rpm for about 60 seconds over RCA-cleaned 22 mm #1 cover slips. The cover slips were then baked in an oven at about 80° C. for about 30 minutes. Chip pieces were washed in ethanol and blown dry before binding to the PDMS layer on the cover slips. The now assembled chips underwent final bake in an oven at about 80° C. for about 2 hours. The yield was about 95%, with the 5% loss being attributed to the dust and debris that are trapped between layers.

The flow channels of the PDMS chip were filled with a mixture of DAPEG and the Pt catalyst at the volumetric ratio between DAPEG and catalyst of about 200:1. Then, the chip was baked in an oven at about 80° C. for about 30 min. The DAPEG mixture was flushed out of the microchannels with high purity water. Alternating layers of poly(ethylene imine) and poly(acrylic acid) were built using about 5 minute feeds of about 20 mg/ml solutions at pH 8. Next, the surface is biotinylated using a kit from Pierce, followed by deposition of Streptavidin Plus at about 1 mg/ml in Trisb.

### EXAMPLE 2

### DNA Sequencing-By-Synthesis Using a Microfluidic Device

This example illustrates the process of DNA sequencing-by-synthesis using a device as described in Example 1.

A microfluidic device fabricated as described in Example 1 was housed in a custom-built aluminum holder, which was placed in a machined attachment to the translation stage of an inverted Olympus IX50 microscope. 23-gauge steel tubes from New England Small Tube Corp. (Litchfield, N.H.) were plugged into the control channel ports of the device. Their other ends were connected through TYGON tubing (Cole-Parmer, Vernon Hills, Ill.) to Lee-valve arrays (Fluidigm Corp. South San Francisco, Calif.) and operated by LabView™ software on a personal computer. The same types of steel tubes and TYGON tubing plumbing were used to supply reagents to the flow channel ports of the device. The microscope was equipped with a mercury lamp (HBO 103 W/2 Osram), an Olympus Plan 10× objective (NA 0.25), an Olympus PlanApo 60x objective (NA 1.4), and a cooled CCD camera SBIG ST-7I (Santa Barbara Instrument Group).

Fluorescence detection was conducted using the following filter sets: (ex D470/40, 500 DCLP, em D535/50) for Alexa Fluor 555, and (ex D540/25, dichroic 565 DCLP, em D605/55) for TAMRA, Lissamine, and Cy3. Both sets were procured from Chroma Technology Corp., Brattleboro.

The following reagents were used for sequencing. DNA1 was an 89-mer biotinylated DNA template (Biotin-5′-(tcatcag)₁₀tcatcACACGGAGGTTCTA-3′; SEQ ID NO: 1) annealed to a 14-mer primer tagged with the Cy3 fluorescent dye (Cy3-5′-TAGAACCTCCGTGT-3′; SEQ ID NO:2). DNA2 was a 99-mer biotinylated DNA template (biotin-5′-(tttgcttcttattc)₆ ttACACGGAGGTTCTA; SEQ ID NO:3) annealed to the same type of primer. All DNA was obtained from Operon Co. (Alameda, Calif.). The buffer was TrisMg which is Tris 10 mM (NaCl 10 mM, MgC12 100 mM), pH 8.

The sequencing feeds contained: A (10 μM dATP-Lis, 2 μM dATP, polymerase), C (10 μM ddCTP-TAMRA, 0.2 μM dCTP, polymerase), G (10 μM ddGTP-TAMRA, 3.3 μM dGTP, polymerase), U (8 μM ddUTP-TAMRA, 28 nM dTTP, polymerase), all in 1× SEQUENASE polymerase reaction buffer with 15 mM DTT. All tagged nucleotides were from PerkinElmer, Boston, Mass. All standard nucleotides were from Boehringer Mannheim (Germany). In all cases, SEQUENASE Version 2.0 DNA polymerase (USB Corp., Cleveland, Ohio) was used for all reactions.

Biotinylated DNA1 at 7 μM in TrisMg was deposited in the flow channel over Streptavidin Plus. 16 polyelectrolyte layers were used. After fluorescence detection confirmed the successful attachment of DNA in the channel, the Cy3 tags were bleached. Next, ddGTP-TAMRA (100 mM in 13 SEQUENASE polymerase reaction buffer with 5 mM DTT) was fed into that channel only, followed by a Trisb flush and fluorescence detection. Then, another solution containing 0.5 U/ml polymerase, but otherwise identical to the first solution, was fed into the same channel, followed by a Trisb flush and fluorescence detection. Later, the same procedure was repeated with the next channel, and so on.

The process was iterated with different feeds in the same chamber, to collect the sequencing data. The net increase in the fluorescent signal after each feed was converted into a corresponding change in fluorophore surface density based on individual reagent calibrations. Next, the same experiment with the same sequence of feeds was repeated in another chamber of the same device, except for withholding the polymerase in all feeds. The similarly extracted data showed the level of nonspecific attachment and was subtracted from the previous data to produce the final results for this experiment (FIG. 6). The measured sequence, GAUG (SEQ ID NO:4), corresponded exactly to the beginning of the known template sequence of CTACTG (SEQ ID NO:5).

These results demonstrate that the microfluidic device system allows for immobilization of DNA such that the target molecule can be subjected to sequencing-by-synthesis reactions.

Although the invention has been described with reference to the above examples, it will be understood that modifications and variations are encompassed within the spirit and scope of the invention. Accordingly, the invention is limited only by the following claims.

What is claimed is:

1. A device, comprising a multi-layer polymeric structure, which comprises:
(a) a graft-copolymer comprising diacrylated polyglycol grafted to a second polymer; and
(b) a plurality of layers of a polyelectrolyte disposed over the graft-copolymer.

2. The device of claim 1, wherein the device is disposed on an optically transparent substrate.

3. The device of claim 2, wherein the device is a microfluidic device.

4. The device of claim 3, wherein the microfluidic device further comprises:
(a) a first layer defining a plurality of first channels;
(b) a second layer bonded to the first layer; and
(c) a sample of a DNA anchored to the inner surface of the first channels,
wherein the inner surface of each first channel is modified to include the multi-layer polymeric structure.

5. The device of claim 1, wherein the multi-layer polymeric structure further comprises biotin conjugated to the layer of the polyelectrolyte.

6. The device of claim 5, further comprising streptavidin disposed over biotin.

7. The device of claim 1, wherein the second polymer is selected from a group consisting of poly(dimethylsiloxane), poly(methylmethacrylate), and poly(urethane).

8. The device of claim 1, wherein the polyelectrolyte comprises alternating layers of a positively charged polymer and a negatively charged polymer.

9. The device of claim 8, wherein the positively charged polymer is selected from poly(ethyleneimine) and poly(allylamine).

10. The device of claim 8, wherein the negatively charged polymer is poly(acrylic acid).

11. The device of claim 2, wherein the optically transparent substrate comprises glass.

12. The device of claim 2, wherein the device is bonded to the optically transparent substrate.

13. The device of claim 12, wherein the device is bonded with poly(dimethylsiloxane), poly(methylmethacrylate), poly(urethane), or a combination thereof.

14. The microfluidic device of claim 4, wherein each first channel has a height of about 10 micrometers.

15. The microfluidic device of claim 4, wherein each first channel has the width of between about 60 and 150 micrometers.

16. The microfluidic device of claim 4, wherein the plurality of the first channels is between 2 and 50.

17. The microfluidic device of claim 4, wherein the second layer further defines a plurality of second channels.

18. The microfluidic device of claim 17, wherein each second channel has a height of about 10 micrometers.

19. The microfluidic device of claim 17, wherein each second channel has a width of between about 60 and 150 micrometers.

20. The microfluidic device of claim 17, wherein the plurality of the second channels is between 2 and 50.

21. A method for fabricating a microfluidic device, comprising:
(a) fabricating a first polymeric structure and a second polymeric structure, each structure defining a plurality of channels;
(b) aligning the first polymeric structure and the second polymeric structure,
wherein the channels in the first polymeric structure are not fluidically connected to the channels in the second polymeric structure, and
wherein the channels in the first polymeric structure and the channels in the second polymeric structure create a valve action;
(c) bonding the first polymeric structure to the second polymeric structure to obtain a fused structure;
(d) bonding the fused structure to an optically transparent substrate; and
(e) modifying the channels in the first polymeric structure by grafting a modifying polymer to the polymer forming the first polymeric structure; to obtain a graft copolymer, and forming a layer of a polyelectrolytes over the graft copolymer,
thereby fabricating the microfluidic device.

22. The method of claim 21, wherein the modifying polymer is diacrylated poly(ethylene glycol).

23. The method of claim 21, further comprising applying a biotin over the layer of the polyelectrolytes.

24. The method of claim 23, further comprising applying streptavidin over biotin.

25. The method of claim 21, wherein forming the layer of the polyelectrolyte comprises applying layers of a positively charged polymer and a negatively charged polymer in alternating manner.

26. The method of claim 25, wherein the positively charged polymer is selected from poly(ethyleneimine) and poly(allylamine).

27. The method of claim 25, wherein the negatively charged polymer is poly(acrylic acid).

28. The method of claim 21, wherein the polymer forming the first polymeric structure and the second polymeric structure is poly(dimethylsiloxane), poly(methylmethacrylate), poly(urethane), or a combination thereof.

29. An assembly, comprising a plurality of devices of claim 1.

30. The device of claim 1, wherein the polyglycol is poly(ethylene glycol).

31. The method of claim 21, wherein the modifying polymer is diacrylated polyglycol.


SEQUENCE LISTING (3 sequences)

SEQ ID NO: 1 - DNA, 70 bp, Artificial sequence
  Synthetic construct template
tcatcagtcatcagtcatcagtcatcagtcatcagtcatcagtcatcagtcatcagtcatcagtcatcag

SEQ ID NO: 2 - DNA, 14 bp, Artificial sequence
  Primer
tagaacctccgtgt

SEQ ID NO: 3 - DNA, 100 bp, Artificial sequence
  Synthetic construct template
tttgcttcttattctttgcttcttattctttgcttcttattctttgcttcttattctttgcttcttattctttgcttcttattcttacacggaggttcta